(19) United States
(12) Patent Application Publication
(10) Pub. No.: US 2005/0287549 A1
(43) Pub. Date: Dec. 29, 2005

(54) Method of genetic testing

(75) Inventors: Keiichi Nagai, Higashiyamato (JP); Kazunori Okano, Tokyo (JP); Hideyuki Noda, Kokubunji (JP); Hiroko Matsunaga, Kodaira (JP); Kiyomi Taniguchi, Kokubunji (JP); Yoshiaki Yazawa, Nishitokyo (JP); Tomoharu Kajiyama, Kodaira (JP)

Correspondence Address: Stanley P. Fisher;Reed Smith LLP, Suite 1400, 3110 Fairview Park Drive, Falls Church, VA, 22042-4503, US

(73) Assignee: Hitachi, Ltd.

(21) Appl. No.: 11/041,456

(22) Filed: Jan. 25, 2005

(30) Foreign Application Priority Data
Jun. 29, 2004 (JP) 2004-191781

(51) Int. Cl. C12Q 1/68

(52) U.S. Cl. 435/6

(57) ABSTRACT

This invention provides a method of genetic testing that enables testing of a plurality of variation sites (SNPs) in a cost-effective and simple manner, allowing realization of genetic diagnosis in clinical settings. The SNP type of the nucleic acid sample is evaluated by: allowing a nucleic acid sample having an anchor sequence at its 5′ end to hybridize to a support having, immobilized on its surface, a probe containing a sequence that is complementary to the target sequence (the SNP region); extending a complementary strand from the probe utilizing the nucleic acid sample as a template; dissociating and removing the nucleic acid sample from the extended probe; extending a complementary strand using the extended probe as a template and a primer having a sequence identical to the anchor sequence; and detecting pyrophosphoric acid generated via the primer extension, based on bioluminescence.

### CLAIM OF PRIORITY

The present application claims priority from Japanese application JP 2004-191781 filed on Jun. 29, 2004, the content of which is hereby incorporated by reference into this application.

### TECHNICAL FIELD

The present invention relates to a technique of genetic testing and more particularly to a technique of genetic testing or diagnosis that is intended to detect a genetic polymorphism or the like in DNA.

### BACKGROUND ART

Up to the present, genomic sequences of a variety of model animals including humans have become available, and effective utilization of such genomic sequences have been actively attempted in a variety of fields, including medical, medicine manufacturing, and other fields. Large-scale analysis of the single nucleotide polymorphisms (SNPs) that are single nucleotide substitutions in genomic sequences has been particularly promoted from the viewpoint of the effectiveness of the inspection of the correlation between a gene and a disease or sensitivity to medicines. If such analysis is able to elucidate the correlation between the individual's SNPs and a disease or sensitivity to medicines, diagnosis of diseases based on the individual's SNPs information or the inspection of sensitivity to medicines would become common. Also, demand for a test that is referred to as “genetic diagnosis” is considered to have become increased.

Unlike the analysis of unknown genes, the targets of genetic diagnosis are known genes or the occurrence of variations thereof. Thus, such tests are preferably carried out in a cost-effective manner, and a variety of methods for carrying them out have been developed. In the case of genetic diagnosis of diseases such as lifestyle-related diseases that are considered to develop due to the combination of a plurality of genes and environmental factors, testing of a plurality of genes in addition to testing of a single gene is critical. Accordingly, a method and an apparatus that enable testing of a plurality of genes in a simple and cost-effective manner have been desired.

A wide variety of methods for detecting SNPs have been proposed. Examples thereof include: the Taqman assay for detecting increased fluorescence upon degradation of a marker probe at the time of PCR amplification (Procedure Natural Academy of Sciences, U.S.A., 88, pp. 7276-7280, 1991); the Invader assay for detecting fluorescence by degrading a quenched fluorescence-labeled probe with the use of a combination of the formation of triple-stranded DNA and an enzyme that recognizes a mismatch (Nature Biotechnology 17, pp. 292-296, 1999); the single strand conformation polymorphisms (SSCP) method for detecting SNPs via separation thereof by gel electrophoresis based on differing electrophoretic mobility due to different higher-order structures caused by a variation-containing DNA strand (Genomics 5, pp. 874-879, 1989); a method wherein a DNA chip is employed (Genomic Research 10, pp. 853-860, 2000); and a method wherein a DNA probe is immobilized on color-coded fine particles, such particles are gathered to prepare a probe array, and the probe array is then used (Science 287, pp. 451-452, 2000). All of these methods are carried out by fluorescence detection utilizing a laser as an excitation light source.

Pyrosequencing that utilizes bioluminescence (Analytical Biochemistry 280, pp. 103-110, 2000) and the bioluminometric assay with modified primer extension reaction (BAMPER) method (Nucleic Acids Research 29, e93, 2001) have been reported as methods for detecting DNA without the use of laser-excited fluorescence.

The BAMPER method enables detection of DNA variation with the utilization of bioluminescence in a simple and cost-effective manner, which had been developed by one of the present inventors and others. In this method, the 3′ end of the DNA probe is generally made to match a variation site of target DNA. In general, when the 3′ end of the primer used for complementary strand synthesis is complementary to the target DNA sequence and completely hybridizes therewith, complementary strand synthesis takes place. When a non-complementary site is present, however, complementary strand synthesis does not take place or is less likely to take place. More specifically, complementary strand synthesis can be regulated by whether or not the 3′ end of the primer matches or does not match, i.e., whether the 3′ end of the primer is complementary or non-complementary to the target DNA. Further, when a type of nucleotide in the vicinity of the 3′ end of the primer differs from the type of nucleotide that is complementary to the target DNA, the level of hybridization in the vicinity of the 3′ end of the primer becomes low. Thus, when the 3′ end of the primer is complementary to the target DNA, complementary strand synthesis is carried out with substantially the same efficiency as that attained when the original primer is used. In contrast, when the 3′ end is not complementary thereto, complementary strand synthesis is not substantially carried out. In the BAMPER method, a primer having such artificial mismatch introduced to the vicinity of the 3′ end is used to carry out complementary strand synthesis, the generated pyrophosphoric acid is converted to ATP, and the bioluminescence induced therefrom is assayed. Thus, the occurrence of complementary strand synthesis, i.e., the occurrence of variation in the target DNA, is detected.

In the BAMPER method, pyrophosphoric acid is generated in accordance with the length of a DNA strand that is extended via complementary strand synthesis. In principle, accordingly, a signal that is attained by this method could be larger by approximately two orders of magnitude than a signal that is attained by pyrosequencing, in which pyrophosphoric acid generated via single nucleotide extension at the time of complementary strand synthesis is detected. In order to accurately distinguish the allele at the SNP site, two types of probes having terminal sequences complementary to each allele are prepared, they are independently allowed to react, and the levels of bioluminescence generated are compared. Thus, the presence of SNP and whether or not such SNP is heterozygous or homozygous can be determined.

Properties that are necessary for practical methods of genetic diagnosis include simplicity, lack of necessity of any expensive apparatus, simple procedures, and feasibility of batch testing of a plurality of test sites. Many techniques that have been developed and employed to date require amplification of DNA or preparation of assay samples for each test object. When the test object has a plurality of target sites, accordingly, such method disadvantageously requires effort, time, and expense. Also, a method that employs fluorescence detection has been problematic in terms of expense due to the necessity of a fluorescence-labeled nucleotide or a probe reagent and an apparatus equipped with a laser. In order to deal with such demands and problems, one of the present inventors and others have proposed the aforementioned BAMPER method, and have produced good outcomes.

Even the BAMPER method, however, required the amplification of the subject DNA via PCR or other means prior to the assay and purification of single-stranded DNA with the use of magnetic beads or the like. Thus, some of present inventors and others improved the BAMPER method and developed a method wherein the sequence of interest and complementary strands specific to the variation of interest are directly synthesized from double-stranded DNA without purifying the template single-stranded DNA to detect pyrophosphoric acid generated, based on the bioluminescence (JP Patent Publication (Kokai) No. 2003-135098 A). In principle, operations covering preparation, testing, and assay of samples can be carried out in a single reaction vessel according to the aforementioned method. Thus, simple and cost-effective SNPs typing can be realized, although a process of degrading pyrophosphoric acid, amplification primers, dNTPs, and the like remaining in the specimen with enzymes is required after the process of amplifying the target region. Although individual reaction can be carried out in a single reaction vessel, a plurality of reaction vessels are required in order to test a plurality of target regions that are necessary for the analysis of multifactorial genetic diseases or haplotypes.

Some of present inventors and others developed a method for simultaneously analyzing samples having a plurality of target regions via simultaneous assay of bioluminescence via the BAMPER method in subcells divided for each target (JP Patent Publication (Kokai) No. 2003-135097 A). In this method, detection sensitivity is enhanced by amplifying the amount of pyrophosphoric acid generated as a result of complementary strand synthesis instead of amplifying the number of target DNA copies. Thus, the issue of side products generated upon PCR amplification is overcome. In this method, however, the extension of the complementary strand is carried out on a solid phase, and this causes the probability of a substrate being in contact with a probe to become lower and the efficiency of extension of the complementary strand to become lower than that attained on a liquid phase.

### DISCLOSURE OF THE INVENTION

The present invention is directed to resolving the problems of conventional methods of genetic testing and to providing a method of genetic testing that is capable of simultaneously testing a plurality of target regions with the utilization of simplified procedures and apparatuses.

According to the present invention, probes corresponding to a plurality of target regions were immobilized on the surface of the solid phase. This eliminated the need for a step of degrading pyrophosphoric acid, amplification primers, and dNTPs remaining in the specimen after the step of amplifying the target regions with enzymes. It also enabled simultaneous detection of a plurality of target regions with a single device.

More specifically, the present invention relates to a method of genetic testing comprising steps of:
allowing a nucleic acid sample having an anchor sequence at its 5′ end to hybridize to a support having, immobilized on its surface, a probe containing a sequence that is complementary to the target sequence;
extending a complementary strand from the probe utilizing the nucleic acid sample as a template;
dissociating and removing the nucleic acid sample from the extended probe synthesized during the above extension of the complementary strand;
extending a complementary strand using the extended probe as a template and a primer having a sequence identical to the anchor sequence; and
detecting pyrophosphoric acid generated via the primer extension, based on bioluminescence.

In the method according to the present invention, the test targets, such as genomic DNA, are subjected to nucleic acid amplification with the use of primers, at least one of which has a common anchor sequence on its 5′ end, thereby obtaining a nucleic acid sample, at least one strand of which has a common sequence (a sequence corresponding to an anchor sequence) on its 5′ end.

Subsequently, the nucleic acid sample having the anchor sequence is applied on the surface of the support having, immobilized thereon, a probe corresponding to the target sequence with the reaction solution for the extension of the complementary strand that contains DNA polymerase and a substrate to extend the complementary strand. Thus, the complementary strand is synthesized only from the probe corresponding to the sequence of the nucleic acid sample on the surface of the support. The extension of the complementary strand from the probe can be carried out under thermal cycle conditions in accordance with a conventional technique.

After the completion of the reaction, the surface of the solid is washed and a denaturing agent is added to convert the extended chain from the probe on the surface of the support to single-stranded DNA. Pyrophosphoric acid, amplification primers, and dNTPs remaining in the specimen after the step of amplifying the test target region are simultaneously removed by the aforementioned washing. A primer having a sequence identical to the anchor sequence, a reaction solution for the extension of the complementary strand that contains DNA polymerase and substrates, and luminous reagents that generates bioluminescence from pyrophosphoric acid generated upon the extension of the complementary strand are added thereto. Thus, detection of the generated bioluminescence enables identification of the probe-immobilized region where the extension of the specific complementary strand took place and identification of the sequence of the nucleic acid sample.

According to the method of the present invention, a step of extending a complementary strand using a primer having a sequence identical to the anchor sequence may be simultaneously carried out with a step of detecting pyrophosphoric acid generated by the extension, based on bioluminescence.

In the method of the present invention, single-stranded DNA may be used instead of double-stranded DNA as an amplification product of genomic DNA that is used as a template for the extension of an immobilized probe. In such a case, one of the primers to be used for nucleic acid amplification is biotin-labeled, the amplification product from the primers is immobilized on the surface of the carrier through avidin via biotin-avidin reactions, and the amplification product is denatured to a single-stranded nucleic acid to obtain a nucleic acid sample consisting of a single-stranded nucleic acid.

In an embodiment, the method of genetic testing according to the present invention is employed for typing of specific variation sites. Specifically, each probe corresponding to a possible sequence at the target variation site is immobilized on the support in a manner such that the probes can be distinguished from each other, and typing of variation of the nucleic acid samples is carried out based on the bioluminescence from a probe-immobilized region. For example, when the target variation is a genetic polymorphism, a probe corresponding to an allele of the polymorphism is immobilized on the support, and typing of a polymorphism of the nucleic acid sample is carried out based on bioluminescence from the probe-immobilized region.

In another embodiment, the method according to the present invention is employed for simultaneous typing of a plurality of variations. In such a case, each probe corresponding to one of a plurality of target variation sites is immobilized on the support in a manner such that the probes can be distinguished from each other, and simultaneous typing of variations in the nucleic acid samples are achieved based on the bioluminescence from the probe-immobilized region. For example, when a plurality of target variations are a plurality of genetic polymorphisms, probes corresponding to alleles of the plurality of polymorphisms are immobilized on the same support in a manner such that the probes can be distinguished from each other, and simultaneous typing of a plurality of polymorphisms in the nucleic acid sample are achieved based on bioluminescence from the probe-immobilized region.

In the case of typing mentioned above, the 3′ end of each probe is designed to correspond to the variation site (the polymorphic site), and specific hybridization and extension of the complementary strand take place only when the nucleic acid sample has a sequence complementary to the sequence at the 3′ end of the probe.

Further, a mismatch may be introduced to a position between the second and the fourth nucleotides from the 3′ end of the probe. This enhances the specificity of hybridization between a probe and a nucleic acid sample and improves the sensitivity of detection.

In a given embodiment, the aforementioned variation is a single nucleotide polymorphism.

Preferably, the probe is immobilized on the support in a manner such that the support is partitioned for a probe-immobilized region. For example, a partition with a height of approximately 1 mm and a width of approximately 2 mm is provided for a probe-immobilized region. This can prevent pyrophosphoric acid that is generated upon the final extension of the complementary strand from diffusive spreading. Thus, luminescence from pyrophosphoric acid is localized and accuracy of detection can be improved.

In the method according to the present invention, the anchor sequence is not particularly limited as long as it is nonspecific for a nucleic acid sample. An example thereof is a poly A sequence.

In the method according to the present invention, the support is preferably present in a vessel having sites for introducing and discharging the nucleic acid sample or a reaction reagent. A light-detecting device for detecting bioluminescence is preferably located in each probe-immobilized region on the support.

Further, a light-guiding path is preferably located between the light-detecting device and the support. Examples of the light-guiding path that can be employed include a rod lens, a spherical lens, and a fiber optic rod.

In general, efficiency of the extension of the complementary strand becomes lower when it takes place on a solid phase than when it takes place on a liquid phase, because the probability such that a substrate would be brought into contact with a probe or the like becomes lower. However, primer extension for generating pyrophosphoric acid is advanced from a liquid phase side instead of from a solid phase in the method according to the present invention. Accordingly, efficiency of extension can be improved and efficiency of pyrophosphoric acid generation can be improved.

According to the present invention, procedures from amplification to assay of genomic DNA in the analysis of a plurality of target regions (for example, SNP sites) can be substantially carried out in a single device. Accordingly, operations thereof are simple, the cost of testing is low, and genetic testing can be realized in clinical settings.

### BRIEF DESCRIPTION OF THE DRAWINGS

FIG. 1 schematically shows the process according to the present invention.

FIG. 2 shows an example of a PCR primer set used in the Examples of the present invention.

FIG. 3 shows the sequence of the probe for SNP identification used in the Examples of the present invention.

FIG. 4 shows an example of a luminescence pattern attained in the present invention.

FIG. 5 shows the constitution of the detector according to the present invention.

FIG. 6 shows the constitution of the chip device according to the present invention.

### BEST MODES FOR CARRYING OUT THE INVENTION

The present invention is hereafter described in detail with reference to the drawings.

1. Anchor Sequence

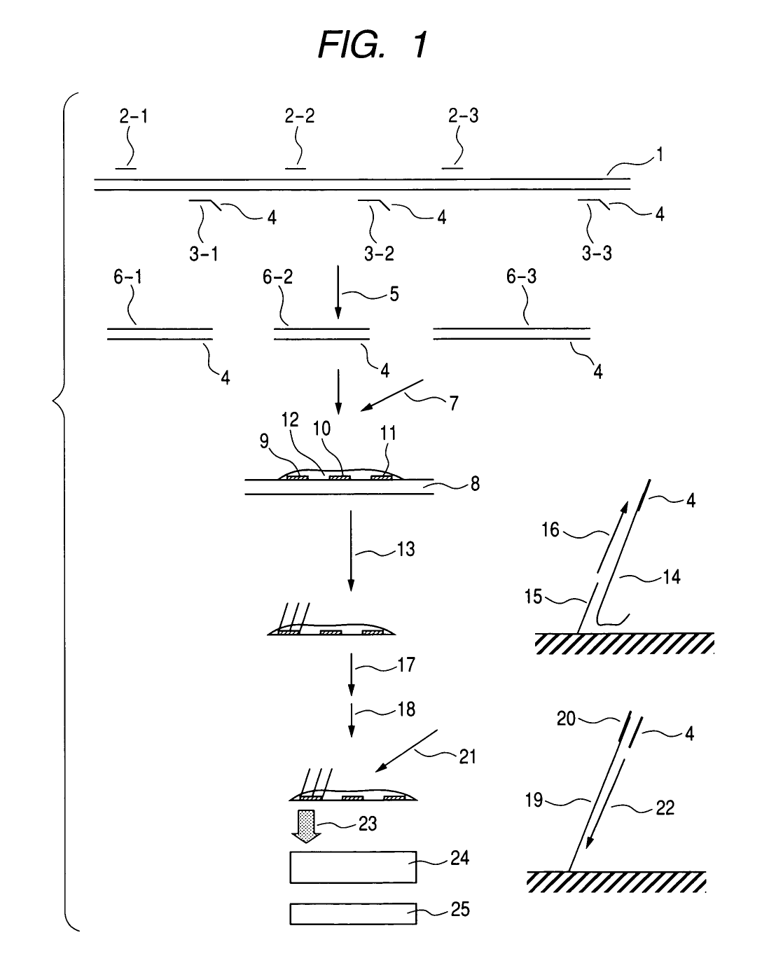

FIG. 1 schematically shows the process of SNP detection according to the present invention. At the outset, genome DNA 1 extracted from a human blood sample or the like is used as a starting material, the PCR primer sets 2 and 3 corresponding to a plurality of gene loci that are targets of SNP assay are used, and a plurality of gene loci are simultaneously amplified by PCR (“5” in FIG. 1).

Each primer set is designed to amplify a nucleic acid fragment containing the target SNP site and having a length of approximately 100 to 1,000 nucleotides, and preferably approximately 150 to 300 nucleotides, on the genome. A common anchor sequence 4 is added to the 5′ end of one member (“3” in the drawing) of a primer set. Thus, the common sequence 4 is introduced to one end of the PCR amplification product The aforementioned anchor sequence is not particularly limited as long as such sequence is template-non-specific and consists of any nucleotide sequence having a length of 10 to 40 nucleotides, and preferably 15 to 25 nucleotides. An example thereof is a poly A sequence.

2. Probe and Support (Chip)

A support (a chip in this case) having, immobilized on its surface, a DNA probe containing a sequence complementary to the target sequence is then prepared. The DNA probe immobilized on the support is an oligonucleotide that has a nucleotide sequence complementary to the target sequence (a variation such as SNP) and is designed to have the 3′ end matching the target SNP site. A mismatch may be introduced to a position between the second and the fourth nucleotides from the 3′ end of the DNA probe, if necessary. Introduction of a mismatch refers to introduction of a nucleotide that is non-complementary to the template sequence or introduction of a nucleotide equivalent (e.g., a spacer) in the probe. In the present invention, the length of the DNA probe is not particularly limited. The length thereof is preferably approximately 10 to 50 nucleotides, and particularly preferably approximately 20 to 30 nucleotides.

Concerning a plurality of target variations (SNPs), the DNA probe is preferably immobilized on a specific site on the chip in a manner such that a probe corresponding to the wild-type 9 or the variant 10 is combined with the control probe 11 having nothing immobilized thereon. The DNA probe can be easily immobilized on the chip in accordance with a conventional technique (for example, Nucleic Acids Research 30, e87, 2002) using a commercially available spotter. Alternatively, the DNA probe may be synthesized on the substrate to prepare a chip.

Partitions may be adequately provided around each DNA probe-immobilized region on the aforementioned chip. For example, provision of a partition with a height of 1 mm and a width of 2 mm can prevent pyrophosphoric acid that is generated upon the final extension of the complementary strand from diffusive spreading. Thus, luminescence from pyrophosphoric acid is localized and accuracy of detection can be improved.

The support is not limited to a chip (made of glass, metal, or plastic), and any solid support, such as a membrane filter, capillary, or bead, can be employed as long as DNA can be immobilized thereon.

3. Extension of Specific Complementary Strand

The PCR amplification product is added dropwise to the chip 8 together with a reaction solution 7 containing a heat-stable enzyme and a substrate for the extension of DNA complementary strands, and the chip is subjected to thermal cycle reaction 13 in a manner such that the DNA probe region on the chip (“12” in the drawing) becomes coated with the reaction solution. In this case, a DNA probe 15 having a 3′ end complementary to the sequence at the SNP site of the PCR amplification product 14 is exclusively extended (“16” in the drawing). After the completion of the reaction, the surface of the solid is washed (“17” in the drawing) and a denaturing agent 18, such as alkali, is added. Thus, the extension product of the DNA probe and the unreacted DNA probe immobilized on the chip surface are both converted to single-stranded DNA 19. In such a case, a sequence complementary to the common anchor sequence 20 is introduced to all the 3′ ends of the extension products of the DNA probe.

4. Bioluminescence and Detection

Subsequently, the DNA-immobilized region of the chip is covered with reaction solution 21 containing a primer having a nucleotide sequence identical to that of the aforementioned anchor sequence, a heat-stable enzyme for extension of the DNA complementary strand, a substrate, and a luminous reagent. Thus, extension of the complementary strand (“22” in the drawing) utilizing the extension product on the chip as a template is simultaneously carried out with the luminescence reaction utilizing the pyrophosphoric acid generated by the extension as a substrate. Apyrase may be added to the reaction solution 21 in order to avoid detection of the diffused pyrophosphoric acid.

In the region where the anchor-sequence-introduced DNA probe has been immobilized, pyrophosphoric acid is generated via extension of the complementary strands. This pyrophosphoric acid is detected as bioluminescence 23 with the aid of luciferin-luciferase. More specifically, pyrophosphoric acid is converted to ATP with the use of ATP sulfurylase. The converted ATP and luciferin are allowed to oxidatively react with the aid of luciferase, and oxyluciferin, pyrophosphoric acid, and other groups of substances are generated. When this generated excited luciferin oxide returns to the normal state, luminescence is generated at around 530 nm. Thus, this luminescence is assayed with the use of a light-detecting device that is capable of distinguishing the luminescent site to evaluate the type of SNP in the genomic DNA.

The method of genetic testing according to the present invention is widely applied to the detection of variations in DNA including the aforementioned SNP as well as the simple presence of DNA. This method can be also applied to detection of the presence of DNA having a specific nucleotide sequence or analysis of gene expression profile via assay of the distribution of cDNA derived from mRNA in the specimen.

### EXAMPLES

The present invention is hereafter described in greater detail with reference to the following examples, although the technical scope of the present invention is not limited thereto.

### Example 1

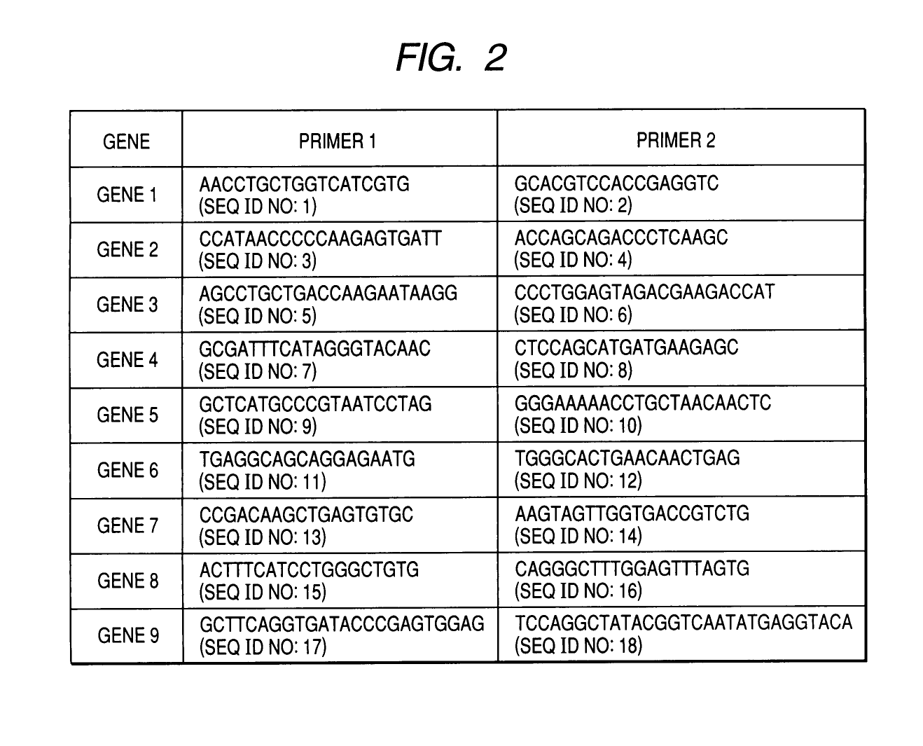

Simultaneous PCR of a plurality of gene loci that employs human genomic DNA as a starting material was carried out in the following manner based on the protocol of the Multiplex PCR Kit (Qiagen). First of all, a total of eighteen types of primers (SEQ ID NOs: 1 to 18) of the primer sets having nine sets of gene-specific sequences as shown in FIG. 2 were diluted to a final concentration of 2 μM with the aid of TE buffer (10 mM Tris-HCl, 1 mM EDTA, pH 8.0). Any anchor sequence may be employed, although it should be nonspecific for the target genomic sequence. In the present example, a poly A sequence consisting of twenty nucleotides was employed as an anchor sequence and it was introduced to the 5′ end of the primer 2 shown in FIG. 2.

Master Mix (20 μl) of the Qiagen Multiplex PCR kit, 4 μl of Q-solution, 2 μl of the primer mix, 11 μl of sterilized water, and 3 μl of human genomic DNA extracted from the blood of an anonymous volunteer were added to a 96-well PCR plate, and these substances were thoroughly mixed with a pipetter. Further, PCR was carried out using a thermal cycler (40 cycles of 95° C. for 15 minutes, 94° C. for 30 seconds, 57° C. for 90 seconds, and 72° C. for 90 seconds, followed by a cycle of 72° C. for 10 minutes, to lower the temperature to 4° C.). Electrophoresis was carried out using 1 μl of the PCR product, and the concentrations and the lengths of nine types of Multiplex PCR products were inspected.

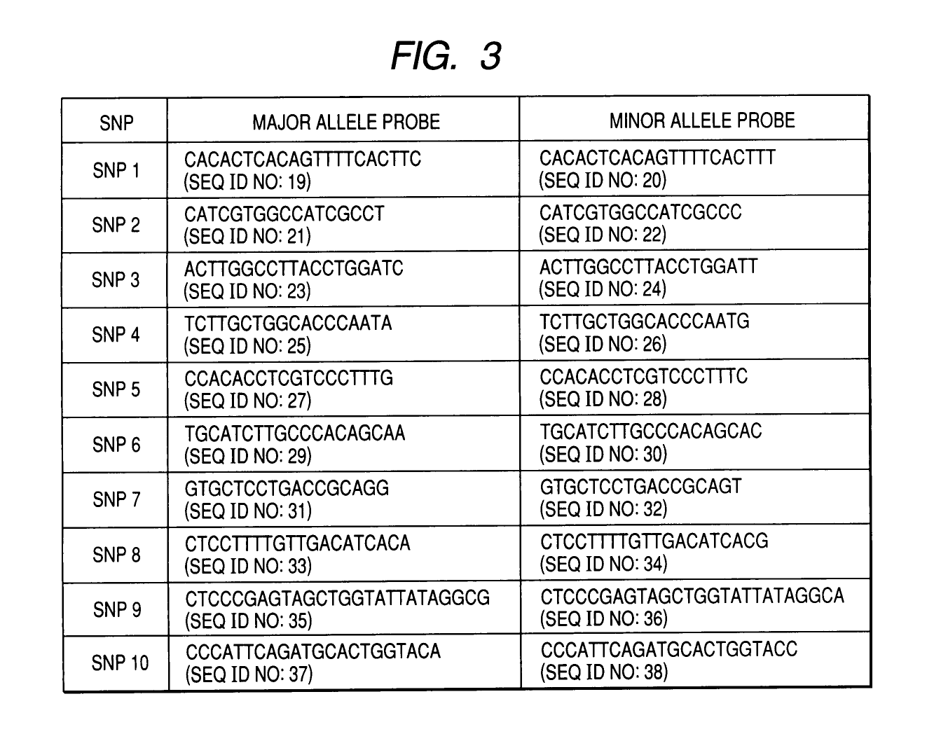

Subsequently, a glass substrate was used as a chip on which probe DNA was to be immobilized, and probe DNA was immobilized on the surface of this glass substrate. Probe DNA can be immobilized on the substrate via a variety of techniques. The present example employed a technique whereby an amino group was introduced into glass substrate with the aid of a silane coupler (3-aminopropyltrimethoxysilane) a maleimide group was introduced into such substrate with the aid of N-(1′-maleimideundocanoxyloxy)succinimide, and an oligo DNA probe having a thiol-modified 5′ end was immobilized thereon (Nucleic Acids Research 30, e87, 2002). The ten sets of immobilized probe sequences (SEQ ID NOs: 19 to 38) for SNP identification are shown in FIG. 3. Since two alleles were present in each SNP, ten probe sets (twenty alleles in total) corresponding to each allele (a major allele and a minor allele) were prepared.

In the extension of the immobilized DNA probe, Taq DNA polymerase (0.05 unit/μl), MgCl₂ (0.15 mM), and dNTP (0.125 mM) were added to the PCR amplification product of genomic DNA, and the mixture was subjected to 5 cycles of 94° C. for 10 seconds, 50° C. for 10 seconds, and 72° C. for 20 seconds.

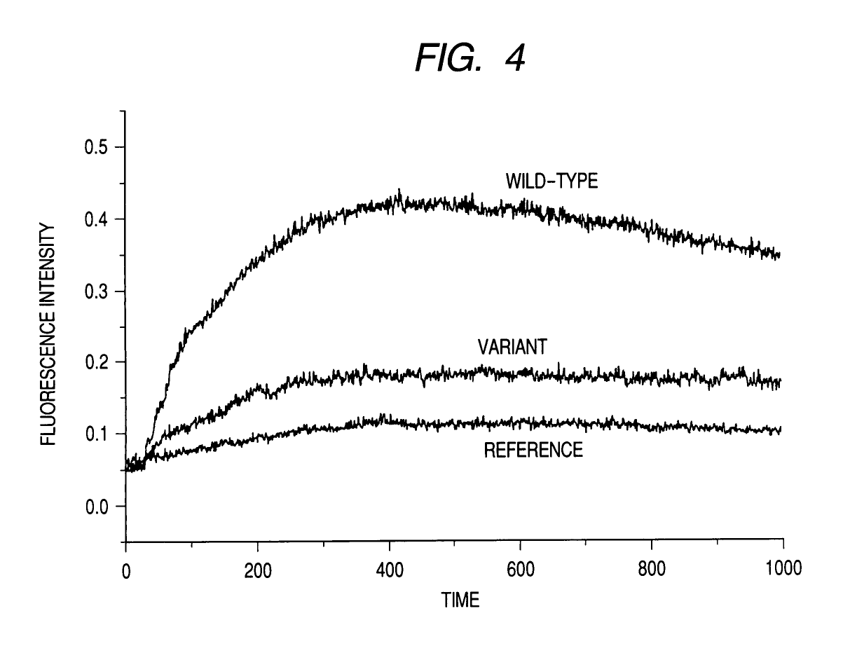

The final extension of the complementary strands from the anchor sequence and the luminescence reaction of generated pyrophosphoric acid were carried out using a DNA primer consisting of a complementary strand of the anchor sequence, the reagent for extension mentioned above that comprises Taq DNA polymerase, MgCl₂, and dNTPs, and luminous reagents (the reagents for the luciferin-luciferase luminescence reaction described in Nucleic Acids Research 29, e93, 2001). An example of the obtained luminescence pattern is shown in FIG. 4. In this example, luminescence from the bottom of the chip was assayed with a CCD camera 25 (C3077-70, Hamamatsu Photonics K.K.) via imaging lens 24.

Single-stranded DNA may be used instead of the aforementioned double-stranded DNA as the amplification product of the genomic DNA used as a template for the extension of the immobilized probe. Among the primer sets shown in FIG. 2, in such a case, a primer having no anchor sequence was labeled with biotin at its 5′ end. Further, PCR was carried out under conditions similar to those described above, the generated amplification product was allowed to react with streptavidin-labeled sepharose (Amersham Biosciences), and the amplification product was trapped on the sepharose. A denaturing solution comprising 0.2 M NaCl was added thereto to recover single-stranded DNA with an anchor sequence that was released in the solution. The neutralized product thereof was used to perform extension of the immobilized DNA probe. Single-stranded DNA may be hybridized to the immobilized DNA probe in an adequate hybridization solution at 42° C. for approximately 30 minutes. After the hybridization, the surface of the chip was washed with sterilized water or the like. The extension reaction may be carried out with the addition of a reaction solution having the same composition as the reaction solution used for the aforementioned double-stranded DNA at 55° C. for approximately 10 minutes.

### Example 2

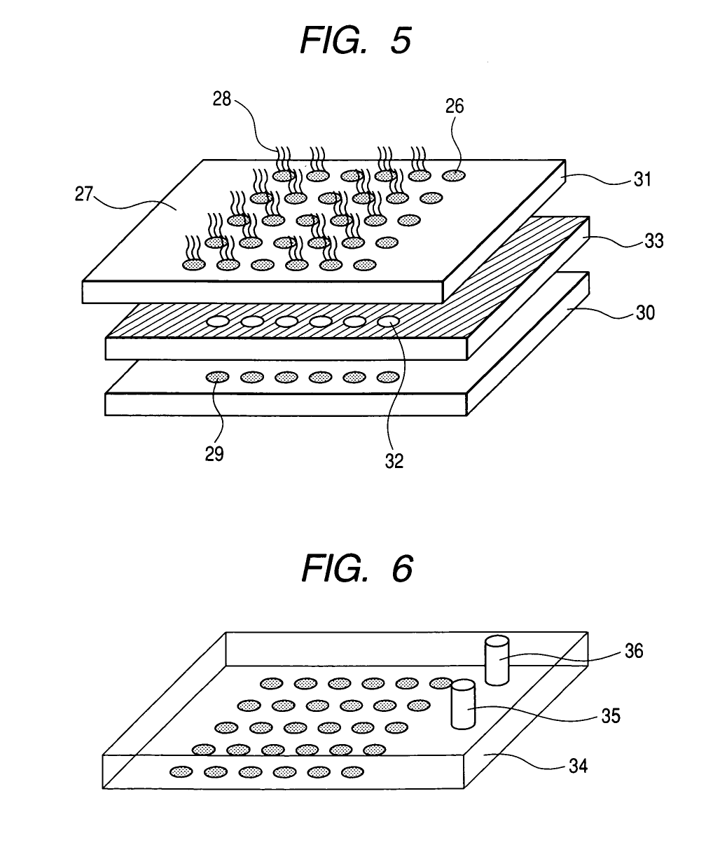

The second example of the present invention is described with reference to FIG. 5. This example concerns an apparatus for detecting luminescence that is suitable for practical application of the present invention. In order to assay the ten SNP sites shown in Example 1, a chip 27 consisting of thirty detection sites 26 (each consisting of three sites) was prepared. The chip was a glass substrate, each site was a circle having a diameter of 1 mm, and DNA probe 28 was immobilized on each site. The distance between spots was 1.5 mm, and such spaces were aligned in 5 columns and 6 rows. Luminescence from 30 luminescent sites was detected from the chip bottom. A detector comprising a plurality of photodiode arrays on a silicon substrate was used. Alignment of each photodiode 29 was the same as that for the detection site on the aforementioned chip.

More specifically, photodiodes having a diameter of 1 mm were aligned in 5 columns and 6 rows at intervals of 1.5 mm. The photodiode array sensor 30 can be prepared in accordance with JP Patent Publication (Kokai) No. 2003-329681. Transparent sheet 31 was brought into close contact with only thirty luminescent sites with a diameter of 1 mm on the lower surface of the chip in order to avoid luminescence crosstalk, and a photodiode array was placed via array 33 of rod lenses 32 constituted by a hyaline cast with a diameter of 2 mm. As a result, a luminescence pattern similar to that shown in FIG. 4 was attained in this example. Luminescence crosstalk can also be reduced with the use of spherical lenses or an optic fiber array instead of the rod lenses.

### Example 3

The third example of the present invention is described with reference to FIG. 6. This example concerns a chip device that is suitable for practical application of the present invention. A chip device 34 that covers the chip regions aligned with the dimensions shown in Example 2 was prepared, and ports 35 and 36 for introducing or discharging reagents and the like were provided. The simultaneous amplification product from the genomic DNA and the reaction solution for the extension of the complementary strand were introduced to the chip device 34, the device was subjected to the thermal cycle as with the case of Example 1, and DNA probes on the chip were subjected to selective extension. After the completion of extension, the reaction solution was discharged through the outlet, a washing liquid and an alkaline solution were successively introduced through the inlet, and such liquids were discharged, thereby converting the extended DNA product on the chip to single-stranded DNA.

Subsequently, a reaction solution similar to that used in Example 1 was added, and the luminescence reaction was carried out with the utilization of extension of the complementary strands from the anchor sequence and the generated pyrophosphoric acid. Luminescence was detected by mounting the chip device 34 on a detector having the constitution as demonstrated in Example 2. Thus, assay of ten SNPs, a total of thirty SNP sites, was carried out in a single operation with the use of a single chip device 34.

Partitions may be provided around the DNA-probe-immobilized region on the aforementioned device. For example, provision of a partition with a height of 1 mm and a width of 2 mm can prevent pyrophosphoric acid generated upon the final extension of the complementary strand from diffusive spreading. Thus, luminescence from pyrophosphoric acid is easily localized and test accuracy can be improved.

### INDUSTRIAL APPLICABILITY

In the field of life sciences, mass-analysis of SNPs has been actively carried out, and correlations between the SNPs of individuals and diseases or medicinal benefits have rapidly been discovered. As a result, tailored medications such as those involving evaluation of the risk of contracting a disease or medicine that is suitable for a given individual could be realized in the near future via assay of individuals' SNPs. Unlike the large-scale SNP typing that is a major technique at present, a process and an apparatus for assaying several tens of SNP sites, which vary among individuals, in a cost-effective and simple manner, are required for genetic diagnosis via tailored medication. The method of genetic testing according to the present invention can be applied to genetic diagnosis via such tailored medication.

Free Text of Sequence Listings

SEQ ID NOs: 1 to 18: description of artificial sequences: synthetic DNA (primers)

SEQ ID NOs: 19 to 38: description of artificial sequences: synthetic DNA (probes)

1. A method of genetic testing comprising steps of:
allowing a nucleic acid sample having an anchor sequence at its 5′ end to hybridize to a support having, immobilized on its surface, a probe containing a sequence that is complementary to the target sequence;
extending the complementary strand from the probe utilizing the nucleic acid sample as a template;
dissociating and removing the nucleic acid sample from the extended probe;
extending a complementary strand using the extended probe as a template and a primer having a sequence identical to the anchor sequence; and
detecting pyrophosphoric acid generated via the primer extension, based on bioluminescence.

2. The method of genetic testing according to claim 1, wherein a step of extending a complementary strand using a primer having a sequence identical to the anchor sequence is simultaneously carried out with a step of detecting pyrophosphoric acid generated by the extension, based on bioluminescence.

3. The method of genetic testing according to claim 1, wherein the nucleic acid sample having an anchor sequence at its 5′ end is obtained via nucleic acid amplification using the primer having an anchor sequence at its 5′ end.

4. The method of genetic testing according to claim 1, wherein the nucleic acid sample is a double-stranded nucleic acid, and the probe extension is carried out under thermal cycle conditions.

5. The method of genetic testing according to claim 1, wherein one of the primers to be used for nucleic acid amplification is biotin-labeled, the amplification product is immobilized on a carrier having avidin immobilized on its surface via biotin-avidin reactions, and the amplification product is denatured to a single-stranded nucleic acid, thereby obtaining a nucleic acid sample consisting of a single-stranded nucleic acid.

6. The method of genetic testing according to claim 1, wherein the target sequence comprises a variation site, each probe corresponding to a possible sequence at the variation site is immobilized on the support in a manner such that all probes can be distinguished from each other, and typing of variation of the nucleic acid samples is carried out based on the bioluminescence from a probe-immobilized region.

7. The method of genetic testing according to claim 6, wherein each probe corresponding to one of a plurality of target variation sites is immobilized on the same support in a manner such that all probes can be distinguished from each other, and simultaneous typing of a variety of variations in the nucleic acid samples is carried out based on the bioluminescence from the probe-immobilized region.

8. The method of genetic testing according to claim 6, wherein the 3′ end of each probe is designed to correspond to the variation site.

9. The method of genetic testing according to claim 8, wherein the probe contains a mismatch in a position between the second and the fourth nucleotides from its 3′ end.

10. The method of genetic testing according to claim 6, wherein the variation is a single nucleotide polymorphism.

11. The method of genetic testing according to claim 1, wherein the probe is immobilized on the support with the provision of partitions for each probe-immobilized region.

12. The method of genetic testing according to claim 1, wherein the anchor sequence is a poly A sequence.

13. The method of genetic testing according to claim 1, wherein the support is present in a vessel having sites for introducing and discharging the nucleic acid sample or a reaction reagent.

14. The method of genetic testing according to claim 1, wherein a light-detecting device for detecting bioluminescence is located in each probe-immobilized region on the support.

15. The method of genetic testing according to claim 14, wherein a light-guiding path is located between the light-detecting device and the support.

16. The method of genetic testing according to claim 14, wherein the light-guiding path is a rod lens, a spherical lens, or a fiber-optic rod.


SEQUENCE LISTING (38 sequences)

SEQ ID NO: 1 - DNA, 18 bp, Artificial Sequence
  Description of Artificial SequenceSynthetic DNA (primer)
aacctgctggtcatcgtg

SEQ ID NO: 2 - DNA, 17 bp, Artificial Sequence
  Description of Artificial SequenceSynthetic DNA (primer)
gcacgtccaccgaggtc

SEQ ID NO: 3 - DNA, 21 bp, Artificial Sequence
  Description of Artificial SequenceSynthetic DNA (primer)
ccataacccccaagagtgatt

SEQ ID NO: 4 - DNA, 18 bp, Artificial Sequence
  Description of Artificial SequenceSynthetic DNA (primer)
accagcagaccctcaagc

SEQ ID NO: 5 - DNA, 22 bp, Artificial Sequence
  Description of Artificial SequenceSynthetic DNA (primer)
agcctgctgaccaagaataagg

SEQ ID NO: 6 - DNA, 22 bp, Artificial Sequence
  Description of Artificial SequenceSynthetic DNA (primer)
ccctggagtagacgaagaccat

SEQ ID NO: 7 - DNA, 20 bp, Artificial Sequence
  Description of Artificial SequenceSynthetic DNA (primer)
gcgatttcatagggtacaac

SEQ ID NO: 8 - DNA, 19 bp, Artificial Sequence
  Description of Artificial SequenceSynthetic DNA (primer)
ctccagcatgatgaagagc

SEQ ID NO: 9 - DNA, 20 bp, Artificial Sequence
  Description of Artificial SequenceSynthetic DNA (primer)
gctcatgcccgtaatcctag

SEQ ID NO: 10 - DNA, 22 bp, Artificial Sequence
  Description of Artificial SequenceSynthetic DNA (primer)
gggaaaaacctgctaacaactc

SEQ ID NO: 11 - DNA, 18 bp, Artificial Sequence
  Description of Artificial SequenceSynthetic DNA (primer)
tgaggcagcaggagaatg

SEQ ID NO: 12 - DNA, 19 bp, Artificial Sequence
  Description of Artificial SequenceSynthetic DNA (primer)
tgggcactgaacaactgag

SEQ ID NO: 13 - DNA, 18 bp, Artificial Sequence
  Description of Artificial SequenceSynthetic DNA (primer)
ccgacaagctgagtgtgc

SEQ ID NO: 14 - DNA, 20 bp, Artificial Sequence
  Description of Artificial SequenceSynthetic DNA (primer)
aagtagttggtgaccgtctg

SEQ ID NO: 15 - DNA, 19 bp, Artificial Sequence
  Description of Artificial SequenceSynthetic DNA (primer)
actttcatcctgggctgtg

SEQ ID NO: 16 - DNA, 20 bp, Artificial Sequence
  Description of Artificial SequenceSynthetic DNA (primer)
cagggctttggagtttagtg

SEQ ID NO: 17 - DNA, 24 bp, Artificial Sequence
  Description of Artificial SequenceSynthetic DNA (primer)
gcttcaggtgatacccgagtggag

SEQ ID NO: 18 - DNA, 29 bp, Artificial Sequence
  Description of Artificial SequenceSynthetic DNA (primer)
tccaggctatacggtcaatatgaggtaca

SEQ ID NO: 19 - DNA, 21 bp, Artificial Sequence
  Description of Artificial SequenceSynthetic DNA (probe)
cacactcacagttttcacttc

SEQ ID NO: 20 - DNA, 21 bp, Artificial Sequence
  Description of Artificial SequenceSynthetic DNA (probe)
cacactcacagttttcacttt

SEQ ID NO: 21 - DNA, 17 bp, Artificial Sequence
  Description of Artificial SequenceSynthetic DNA (probe)
catcgtggccatcgcct

SEQ ID NO: 22 - DNA, 17 bp, Artificial Sequence
  Description of Artificial SequenceSynthetic DNA (probe)
catcgtggccatcgccc

SEQ ID NO: 23 - DNA, 19 bp, Artificial Sequence
  Description of Artificial SequenceSynthetic DNA (probe)
acttggccttacctggatc

SEQ ID NO: 24 - DNA, 19 bp, Artificial Sequence
  Description of Artificial SequenceSynthetic DNA (probe)
acttggccttacctggatt

SEQ ID NO: 25 - DNA, 18 bp, Artificial Sequence
  Description of Artificial SequenceSynthetic DNA (probe)
tcttgctggcacccaata

SEQ ID NO: 26 - DNA, 18 bp, Artificial Sequence
  Description of Artificial SequenceSynthetic DNA (probe)
tcttgctggcacccaatg

SEQ ID NO: 27 - DNA, 18 bp, Artificial Sequence
  Description of Artificial SequenceSynthetic DNA (probe)
ccacacctcgtccctttg

SEQ ID NO: 28 - DNA, 18 bp, Artificial Sequence
  Description of Artificial SequenceSynthetic DNA (probe)
ccacacctcgtccctttc

SEQ ID NO: 29 - DNA, 19 bp, Artificial Sequence
  Description of Artificial SequenceSynthetic DNA (probe)
tgcatcttgcccacagcaa

SEQ ID NO: 30 - DNA, 19 bp, Artificial Sequence
  Description of Artificial SequenceSynthetic DNA (probe)
tgcatcttgcccacagcac

SEQ ID NO: 31 - DNA, 17 bp, Artificial Sequence
  Description of Artificial SequenceSynthetic DNA (probe)
gtgctcctgaccgcagg

SEQ ID NO: 32 - DNA, 17 bp, Artificial Sequence
  Description of Artificial SequenceSynthetic DNA (probe)
gtgctcctgaccgcagt

SEQ ID NO: 33 - DNA, 20 bp, Artificial Sequence
  Description of Artificial SequenceSynthetic DNA (probe)
ctccttttgttgacatcaca

SEQ ID NO: 34 - DNA, 20 bp, Artificial Sequence
  Description of Artificial SequenceSynthetic DNA (probe)
ctccttttgttgacatcacg

SEQ ID NO: 35 - DNA, 26 bp, Artificial Sequence
  Description of Artificial SequenceSynthetic DNA (probe)
ctcccgagtagctggtattataggcg

SEQ ID NO: 36 - DNA, 26 bp, Artificial Sequence
  Description of Artificial SequenceSynthetic DNA (probe)
ctcccgagtagctggtattataggca

SEQ ID NO: 37 - DNA, 22 bp, Artificial Sequence
  Description of Artificial SequenceSynthetic DNA (probe)
cccattcagatgcactggtaca

SEQ ID NO: 38 - DNA, 22 bp, Artificial Sequence
  Description of Artificial SequenceSynthetic DNA (probe)
cccattcagatgcactggtacc